(19) United States
(12) Patent Application Publication
(10) Pub. No.: US 2014/0178894 A1
(43) Pub. Date: Jun. 26, 2014

(54) CULTURE MEDIUM SUITABLE FOR THE CULTURE OF UNDIFFERENTIATED CELLS

(71) Applicants: Mohamed Bentires-Alj, Kembs-Loechle (FR); Stephan Duss, Therwil (CH)

(72) Inventors: Mohamed Bentires-Alj, Kembs-Loechle (FR); Stephan Duss, Therwil (CH)

(73) Assignee: NOVARTIS FORSCHUNGSSTIFTUNG, ZWEIGNIEDERLASSUNG, Basel (CH)

(21) Appl. No.: 14/113,020

(22) PCT Filed: Apr. 20, 2011
(86) PCT No.: PCT/EP12/57097

§ 371 Date: Feb. 28, 2014

(30) Foreign Application Priority Data
Apr. 20, 2011 (EP) 11163177.6

(51) Int. Cl. G01N 33/50

(52) U.S. Cl. CPC G01N 33/5044
USPC 435/7.4; 435/404; 435/402

(57) ABSTRACT

The present application relates to a composition comprising the components listed in Table I in an amount that will lead to the respective concentration specified in Table I for each of said component when said composition is diluted with water into 1× medium, said composition being particularly suitable to generate a chemically-defined medium suitable for culturing cells, especially primary cells, maintaining their proliferation and differentiation potential.

### FIELD OF THE INVENTION

The present invention relates to a culture medium suitable for the long- or short-term in vitro culture of undifferentiated cells.

### BACKGROUND OF THE INVENTION

Each year, breast cancer is diagnosed in over one million women worldwide and more than 400,000 lives are lost to this disease. Although some patients do well after surgery and initial treatment, drug resistance often occurs and tumors relapse. New therapies are likely to arise only from a more thorough understanding of this largely incurable disease.

Breast cancer is a progressive and heterogeneous disease. The heterogeneity is not only observed in tumors from different patients (intertumoral) but also within the same tumor (intratumoral) (Marusyk A and Polyak K, Biochim Biophys Acta 2010). Breast cancer arises in the epithelial cells of the gland and its heterogenous nature depends on the differentiation state of the cell of origin of cancer, the transforming oncogenic events and the microenvironement. It is noteworthy that depending on the differentiation state of the cells in which the cancer originates, the same oncogenic events can evoke tumors with distinct histotypes (Weinberg and Rangarajan, Cancer Cell 2004; Ince et al. Cancer Cell 2007). Indeed, oncogenic transformation of human mammary epithelial cells (HMECs) grown in a culture medium called MEGM generated nonmetastatic squamous cell carcinomas when injected into immunodeficient mice. This breast tumor histotype is rarely (<1%) found in human breast cancers (Elenbaas, Genes Dev 2001). In contrast, human breast epithelial cells isolated from normal tissue, grown in a different culture medium called WIT and transformed with the same set of oncogenes as in Elenbaas et al. 2001, formed metastatic tumors that resemble adenocarcinomas of the breast when injected into immunodeficient mice (Ince et al. Cancer Cell 2007). Given that both studies used the same set of oncogenes, the different histotypes seen in the tumor xenografts was a consequence of changes in the differentiation state of the original cells due to differences in the culture medium. Therefore, it is fundamental to understand the cellular hierarchy of the breast and to control the differentiation state of breast cells when studying them ex vivo.

Interactions of stem cells and the niche are essential for tissue homeostasis by controlling stem cell quiescence and stem cell activation. Recent evidence implies that aberrant stem cell/niche interactions are involved in tumourigenesis and tumor progression (see e.g., Brisken C and Duss S. Stem Cell Rev. 2007). Experiments that functionally describe the cellular compartments controlling self-renewal and quiescence of breast stem- and progenitor cells have not been reported, leaving the composition of the breast stem cell niche unknown. One of the reasons for this lack of knowledge is the absence of a culture medium suitable for the culture of undifferentiated cells.

Therefore, there is a need in the art for a culture medium for the culture of undifferentiated cells, i.e. a culture medium that would maintain the differentiation potential of stem/progenitor cells in in vitro culture.

### SUMMARY OF THE INVENTION

Through their work, the present inventors surprisingly succeeded in defining a culture medium that allows for the maintenance of the differentiation and proliferation potential of stem/progenitor cells

The present invention thus encompasses a composition comprising the components listed in Table I below in an amount that will lead to the respective concentration specified in Table I for each of said component when said composition is diluted with water into 1× medium. In one embodiment, said composition comprises the components in an amount that will lead to the respective concentration specified in Table II below for each of said component when said composition is diluted with water into 1× medium. In one embodiment, said composition is a chemically-defined medium suitable for culturing cells. Said chemically-defined medium can be suitable for culturing primary cells.

In one embodiment, the composition of the invention is in a concentrated form, wherein the concentration of all the components is increased by about the same factor.

In a further embodiment, the composition of the invention further comprises a solid support coated with a matrix. Said matrix can comprise laminins and collagens. For instance said matrix can comprise Matrigel® and collagen type I. In one embodiment, said support is coated with a composition comprising 10-20% of Matrigel® and about 10 μg/ml of collagen type I. In some embodiments, the solid support is a mesh.

The present invention also encompasses a method of culturing cells comprising the step of placing cells in contact with medium made with a composition of the invention.

The present invention further encompasses a method of screening for agents influencing the differentiation of undifferentiated cells comprising the steps of contacting undifferentiated cells cultured in a medium made with a composition of the invention with a candidate agent; and comparing the differentiation state of the previously undifferentiated cells after said contact with said candidate agent with the differentiation state of undifferentiated cells cultured in a medium made with a composition of the invention which have not been contacted with said agent, wherein a difference in the differentiation state of the cells indicates that the agent is an agent influencing the differentiation of undifferentiated cells. Said comparison can be done by comparing the expression profile of the cell populations.

The present invention further encompasses a kit comprising a composition according to any of claims 1 to 10, wherein some of the components are present in one of more containers.

TABLE I

| Component | mg/L 1x medium |
|---|---|
| (+)-Biotin (Vitamin B7) | 0.01-0.02 |
| 17-beta estradiol | 0.0001-0.0005 |
| 2-Deoxy-D-ribose | 0.2-0.3 |
| 2-Methyl-1,4-naphtoquinone | 0.005-0.015 |
| 4-Aminobenzoicacid | 0.02-0.03 |
| Adenine sulfate | 1-10 |
| Adenosine-5-monophosphoricacid H2O | 0.05-0.2 |
| Adenosine-5-triphosphatedisodiumsalt H2O | 0.1-1 |
| Albumin, bovine | 0-100 |
| Ascorbic acid (Vitamin C) | 0.01-0.03 |
| betamercaptoethanol | 1-5 |
| CaCl2 2H2O | 50-150 |
| Catalase | 0.01-0.05 |
| Cholesterol | 0.05-0.2 |
| Cholinechloride | 1-15 |
| Cobalamine (VitaminB12) | 0.2-1.2 |
| Corticosterone | 0.01-0.05 |
| CuSO4 5H2O | 0.0005-0.005 |
| D(−)-Ribose | 0.1-0.5 |
| D(+)Galactose | 0.1-0.5 |
| D(+)-Glucose | 500-2000 |
| DL-α-Tocopherol (Vitamin E) | 1-5 |
| DL-α-Tocopherol Acetate (Vitamin E) | 1-5 |
| D-α-Tocopherol Succinate (Vitamin E) | 0-0.01 |
| D-Pantothenic acid calcium salt | 0.1-0.5 |
| EGF | 0.01-0.05 |
| Ergocalciferol (VitaminD2) | 0.01-0.02 |
| Ethanolamine | 0.01-0.05 |
| Fe(NO3)3 9H2O | 0.1-0.5 |
| FeSO4 7H2O | 0.1-1 |
| Folic acid | 0.1-0.2 |
| Glutathione (reduced) | 0.01-0.03 |
| Glycine | 10-50 |
| Guanine hydrochloride | 0.1-0.2 |
| HEPES | 2000-5000 |
| Hypoxanthine | 1-4 |
| Insulin | 0.05-0.1 |
| KCl | 100-500 |
| L-4-Hydroxyproline | 1-10 |
| L-Alanine | 0-30 |
| L-alanyl-L-glutamine | 100-1000 |
| L-Arginine HCl | 0-200 |
| L-Arginine monohydrochloride | 0-100 |
| L-Asparagine H2O | 1-10 |
| L-Aspartic acid | 5-50 |
| L-Carnitine | 0.01-0.1 |
| L-Cysteine HCl H2O | 1-30 |
| L-Cystine | 5-20 |
| L-Glutamic acid | 10-100 |
| L-Glutamine | 0-100 |
| L-Glutathione reduced | 0.01-0.05 |
| L-HistidineHCl H2O | 10-50 |
| Linoleic Acid | 1-5 |
| Linolenic Acid | 1-5 |
| L-Isoleucine | 1-20 |
| L-Leucine | 10-50 |
| L-Lysine monohydrochloride | 10-100 |
| L-Methionine | 1-20 |
| L-Phenylalanine | 1-30 |
| L-Proline | 10-50 |
| L-Serine | 5-50 |
| L-Threonine | 10-50 |
| L-Tryptophan | 1-10 |
| L-Tyrosine | 10-50 |
| L-Valine | 5-50 |
| MgCl2 6H2O | 10-100 |
| MgSO4 7H2O | 50-200 |
| myo-Inositol | 1-20 |
| Na2HPO4 | 50-100 |
| NaCl | 5000-15000 |
| NaHCO3 | 100-1500 |
| Niacinamide | 0.01-0.03 |
| Nicotinamide | 0.01-0.02 |
| Nicotinic acid | 0.01-0.02 |
| Phenol Red | 0-20 |
| Progesterone | 0.01-0.02 |
| Putrescine | 0.1-1 |
| Pyridoxal hydrochloride (VitaminB6) | 0-0.05 |
| Pyridoxine HCl (VitaminB6) | 0.01-0.05 |
| Pyruvic Acid Na | 10-100 |
| Retinol (Vitamin A1) | 0.1-0.5 |
| Retinyl Acetate (Vitamin A) | 0.1-0.5 |
| Riboflavin (Vitamin B2) | 0.01-0.05 |
| Sodium Selenite | 0.0001-0.0005 |
| Sodium acetate anhydrous | 10-50 |
| Superoxide Dismutase | 0.01-0.1 |
| T3 (Triodo-l-Thyronine) | 0.00001-0.0001 |
| ThiamineHCl (VitaminB1) | 0.1-0.3 |
| Thioctic Acid | 0.05-0.2 |
| Thymidine | 0.1-0.5 |
| Thymine | 0.1-0.3 |
| Transferrin | 0.05-0.2 |
| Tween80 | 1-20 |
| Uracil | 0.1-0.2 |
| Xanthine | 0.1-0.2 |
| ZnSO4 7H2O | 0.1-1 |

TABLE II

| Component | mg/L 1x medium |
|---|---|
| (+)-Biotin (Vitamin B7) | 0.01365 |
| 17-beta estradiol | 0.0003 |
| 2-Deoxy-D-ribose | 0.25 |
| 2-Methyl-1,4-naphtoquinone | 0.01 |
| 4-Aminobenzoicacid | 0.025 |
| Adeninesulfate | 5 |
| Adenosine-5-monophosphoricacid H2O | 0.125 |
| Adenosine-5-triphosphatedisodiumsalt H2O | 0.5 |
| Albumin, bovine | 50 |
| Ascorbic acid (VitaminC) | 0.025 |
| betamercaptoethanol | 4.47 |
| CaCl2 2H2O | 114.8 |
| Catalase | 0.05 |
| Cholesterol | 0.1 |
| Cholinechloride | 7.23 |
| Cobalamine (VitaminB12) | 0.68 |
| Corticosterone | 0.04 |
| CuSO4 5H2O | 0.00125 |
| D(−)-Ribose | 0.25 |
| D(+)Galactose | 0.3 |
| D(+)-Glucose | 1401 |
| DL-α-Tocopherol (Vitamin E) | 2 |
| DL-α-Tocopherol Acetate (Vitamin E) | 2 |
| D-α-Tocopherol Succinate (Vitamin E) | 0.005 |
| D-Pantothenicacid calciumsalt | 0.245 |
| EGF | 0.02 |
| Ergocalciferol (VitaminD2) | 0.05 |
| Ethanolamine | 0.02 |
| Fe(NO3)3 9H2O | 0.36 |
| FeSO4 7H2O | 0.417 |
| Folicacid | 0.665 |
| Glutathione (reduced) | 0.02 |
| Glycine | 28.755 |
| Guaninehydrochloride | 0.15 |
| HEPES | 3574.56 |
| Hypoxanthine | 2.19 |
| Insulin | 0.08 |
| KCl | 312 |
| L-4-Hydroxyproline | 5 |
| L-Alanine | 17 |
| L-alanyl-L-glutamine | 434.44 |
| L-ArginineHCl | 105.5 |
| L-Argininemonohydrochloride | 35 |
| L-Asparagine H2O | 7.505 |
| L-Asparticacid | 21.65 |
| L-Carnitine | 0.04 |
| L-CysteineHCl H2O | 17.5 |
| L-Cystine | 10 |
| L-Glutamicacid | 40.76 |
| L-Glutamine | 73 |
| L-Glutathionereduced | 0.025 |
| L-HistidineHCl H2O | 21.42 |
| Linoleic Acid | 2 |
| Linolenic Acid | 2 |
| L-Isoleucine | 11.97 |
| L-Leucine | 36.55 |
| L-Lysinemonohydrochloride | 53.25 |
| L-Methionine | 9.74 |
| L-Phenylalanine | 14.98 |
| L-Proline | 37.25 |
| L-Serine | 17.75 |
| L-Threonine | 20.95 |
| L-Tryptophan | 6.02 |
| L-Tyrosine | 23.89 |
| L-Valine | 18.35 |
| MgCl2 6H2O | 61.5 |
| MgSO4 7H2O | 100 |
| myo-Inositol | 9.025 |
| Na2HPO4 | 71.02 |
| NaCl | 7799.5 |
| NaHCO3 | 763 |
| Niacinamide | 0.0185 |
| Nicotinamide | 0.0125 |
| Nicotinicacid | 0.0125 |
| Phenol Red | 10.65 |
| Progesterone | 0.0126 |
| Putrescine | 0.4 |
| Pyridoxalhydrochloride (VitaminB6) | 0.025 |
| PyridoxineHCl (VitaminB6) | 0.031 |
| Pyruvic Acid Na | 55 |
| Retinol (Vitamin A1) | 0.2 |
| Retinyl Acetate (Vitamin A) | 0.27 |
| Riboflavin (Vitamin B2) | 0.025 |
| Sodium Selenite | 0.00032 |
| Sodiumacetateanhydrous | 25 |
| Superoxide Dismutase | 0.05 |
| T3 (Triodo-l-Thyronine) | 0.00004 |
| ThiamineHCl (VitaminB1) | 0.18 |
| Thioctic Acid | 0.105 |
| Thymidine | 0.365 |
| Thymine | 0.15 |
| Transferrin | 0.1 |
| Tween80 | 10 |
| Uracil | 0.15 |
| Xanthine | 0.15 |
| ZnSO4 7H2O | 0.4315 |

Note:
the concentrations listed in the above Tables are not absolute and invariable. Since different cell types may have different growth needs, it is contemplated that generally, a 2-10 fold variation (increase or decrease) for each value is an acceptable range of concentration. Some components may tolerate an even larger variation of final concentration. Further optimization can be achieved using these starting concentrations.

These and other aspects of the present invention should be apparent to those skilled in the art, from the teachings herein.

### BRIEF DESCRIPTION OF THE DRAWINGS

The following drawings are illustrative of embodiments of the invention and are not meant to limit the scope of the invention as encompassed by the claims.

FIG. 1: Culture medium according to the invention allows optimal propagation of mammary epithelial progenitor cells and increases luminal epithelial differentiation and proliferation.

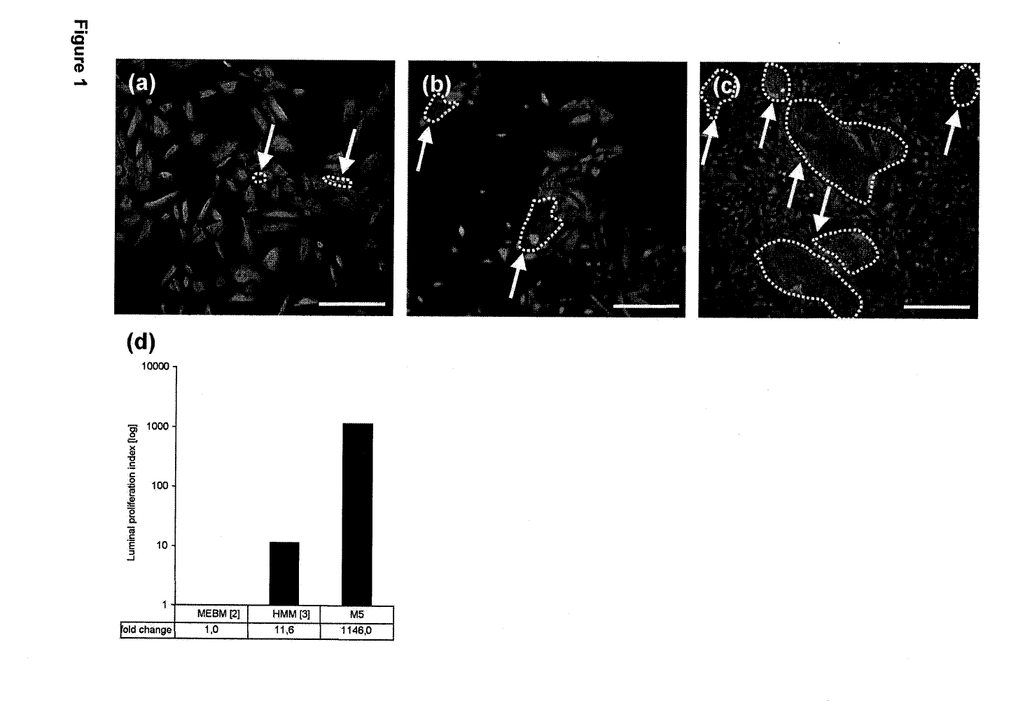

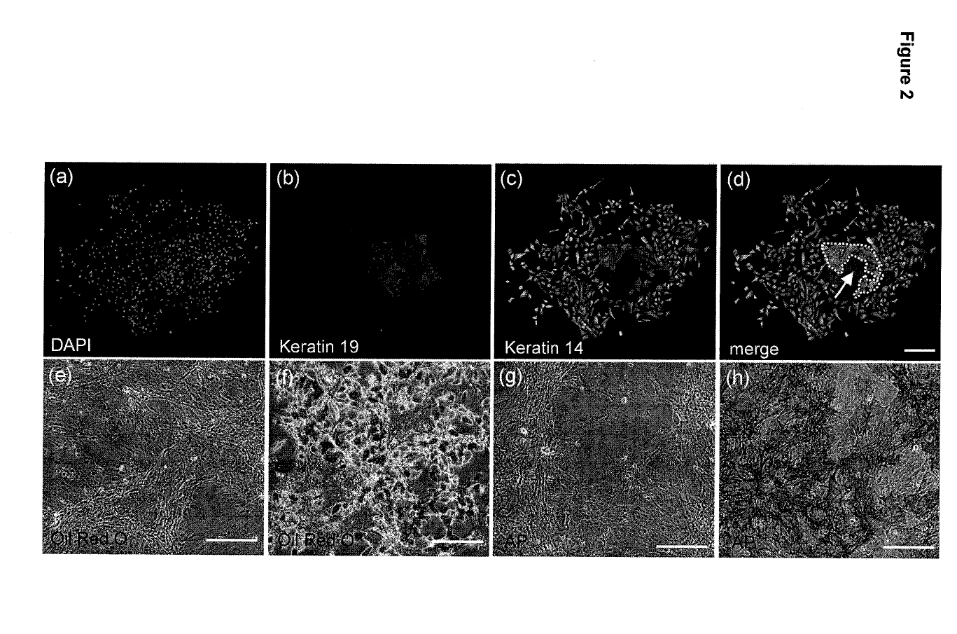

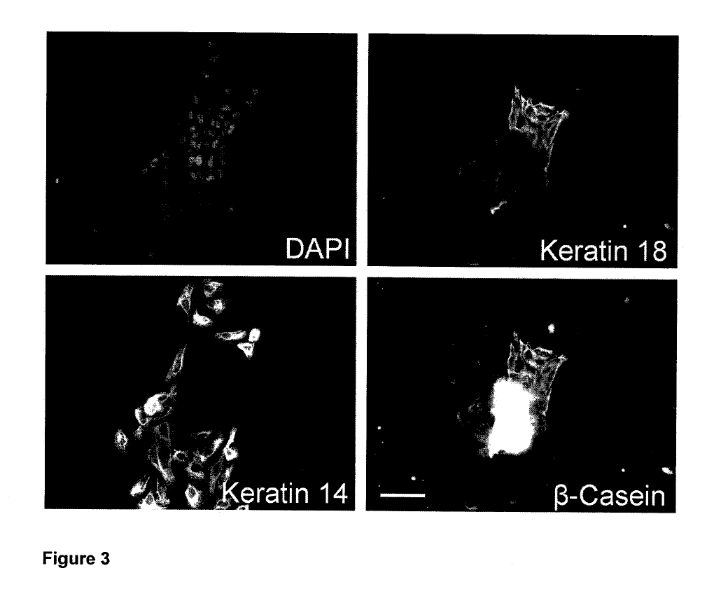

Immunofluorescent staining for the luminal marker Keratin 18 (K18) and the myoepithelial marker Keratin 14 (K14) shows that primary epithelial breast cells mostly differentiate into myoepithelial cells after 28 days cell culture in MEBM (a) and HMM (b). In contrast the medium of the invention allows for differentiation of both luminal and myoepithelial cells (c). In addition quantification of the area covered by K18 positive luminal cells in the three conditions indicates a dramatic increase in luminal differentiation (1000 and 100 fold respectively) and proliferation in the culture medium of the invention (d). Dotted lines and arrows designate the areas covered by K18 positive luminal cells. Scale bars 100 μm.

FIG. 2: Breast epithelial progenitor/stem cells and mesenchymal stem cells propagated in the medium of the invention undergo correct terminal differentiation in differentiating cell culture conditions.

(a-d) Single cells derived from mammospheres maintained in the culture medium of the invention can differentiate in mixed colonies comprising luminal and myoepithelial differentiated breast epithelial cells. Immunofluorescent stainings of luminal Keratin 19 (K19) and myoepithelial Keratin 14 (K14) are shown. Mixed colonies derive from breast progenitor/stem cells and demonstrate that mammospheres grown in the medium of the invention contain breast progenitor/stem cells. Double positive cells (arrow and dotted lines in d) indicate that breast progenitor/stem cells are also maintained under differentiating 2D cell culture conditions. DAPI stain (a) shows cell nuclei. (e-h) Mesenchymal stem cells cultured in adipogenic or osteogenic media can differentiate in adipocytes (f) and osteocytes (h) respectively as compared to the MSC maintained in control medium (e.g). Cell type specific Oil red O (dark bordered droplets and bright areas, e-f) and Alkaline Phosphatase (AP) staining (dark shading, g-h) are shown. Scale bars 100 μm.

FIG. 3: The medium according to the invention supplemented with alveolar differentiation inducing factors allows for the differentiation of beta casein secreting cells. Thus tri-potent epithelial progenitor cells can be maintained and differentiated in a medium according to the present invention.

Breast epithelial cells maintained in the medium according to the invention can differentiate in both luminal Keratin 18 positive (b) and myoepithelial Keratin 14 (c) positive mammary epithelial cell types. Upon extracellular matrix (ECM) and HIP (Hydrocortisone, Insulin and Prolactin) stimulation they can further differentiate into milk producing alveolar cells as indicated by beta casein (β-casein) staining (d). DAPI stain (a) shows cell nuclei. Scale bar: 50 μm.

### DETAILED DESCRIPTION OF THE INVENTION

Through their work, the present inventors surprisingly succeeded in defining a culture medium that allow for the maintenance of the differentiation and proliferation potential of stem/progenitor cells

The present invention thus encompasses a composition comprising the components listed in Table I above in an amount that will lead to the respective concentration specified in Table I for each of said component when said composition is diluted with water into 1× medium. In one embodiment, said composition comprises the components in an amount that will lead to the respective concentration specified in Table II above for each of said component when said composition is diluted with water into 1× medium. In one embodiment, said composition is a chemically-defined medium suitable for culturing cells. Said chemically-defined medium can be suitable for culturing primary cells.

In one embodiment, the composition of the invention is in a concentrated form, wherein the concentration of all the components is increased by about the same factor.

In a further embodiment, the composition of the invention further comprises a solid support coated with a matrix. Said matrix can comprise laminins and collagens. For instance said matrix can comprise Matrigel® and collagen type I. In one embodiment, said support is coated with a composition comprising 10-20% of Matrigel® and about 10 μg/ml of collagen type I. In some embodiments, the solid support is a mesh.

The present invention also encompasses a method of culturing cells comprising the step of placing cells in contact with medium made with a composition of the invention.

The present invention further encompasses a method of screening for agents influencing the differentiation of undifferentiated cells comprising the steps of contacting undifferentiated cells cultured in a medium made with a composition of the invention with an candidate agent; and comparing the differentiation state of the previously undifferentiated cells after said contact with said candidate agent with the differentiation state of undifferentiated cells cultured in a medium made with a composition of the invention which have not been contacted with said agent, wherein a difference in the differentiation state of the cells indicates that the agent is an agent influencing the differentiation of undifferentiated cells. Said comparison can be done by comparing the expression profile of the cell populations.

The present invention further encompasses a kit comprising a composition according to any of claims 1 to 10, wherein some of the components are present in one of more containers.

These and other aspects of the present invention should be apparent to those skilled in the art, from the teachings herein.

The following definitions are provided to facilitate understanding of certain terms used throughout this specification.

Primary cells isolated and cultured using the methods and media of the invention can remain and/or proliferate in an undifferentiated state for many weeks (at least about 4 weeks, and usually more than about 15 weeks) or through many population doublings (PD; at least about 15 PD, usually more than 35 PD) without senescence or detectable genetic alterations. These cells can also be induced to undergo differentiation by changing media and culture conditions. In the case of mammary epithelial progenitors, the cells can differentiate in 3-D culture into epithelial cells of luminal phenotype characterized by luminal epithelial cell marker expression.

In certain embodiments of the invention, “about” or “approximately” refers to a number that varies by up to 5%, or in other embodiments up to 10%, and in other embodiments up to 25%, from the number being referred to. In a specific embodiment, the tissue culture medium of the invention is a chemically defined serum-free medium that is substantially free of animal serum and/or tissue or organ extracts (e.g. Bovine Pituitary Extract).

As used herein, the term “serum-free” refers to the fact that the medium contains essentially no serum. In certain embodiments, there is 0% (completely free), or less than about 0.001%, 0.005%, 0.01%, 0.025%, 0.05%, 0.1%, 1.0%, or 10.0% total serum in the subject medium. The most common types of serums include: various forms of bovine serum (calf serum, fetal bovine serum, bovine calf serum, donor bovine calf serum, newborn bovine calf serum, etc.), horse serum and human serum.

In certain embodiments, there is 1-5% total serum in the subject medium. Such medium may be used for differentiating breast epithelial cells, as well as for propagating other cell types, such as mesenchymal cells.

“Chemically defined” means the structures, chemical formulae, and the percentage of the various individual components within a chemical composition are known or can be defined. Various tissue extracts, such as bovine pituitary extracts, are not chemically defined, at least partly because not all individual components of the extract are known. For those known components, the amount and the relative percentages of the various components could (and usually do) vary from one batch to another. This is partly caused by the fact that individual animals may have inherently different levels of various chemical compositions, even in the same tissue, depending on such factors a general health, nutrition, mood, pathological infections, trauma, etc.

In certain embodiments, the medium of the instant invention does not contain any animal serum products prepared for tissue culture purposes. Nor does it contain any tissue extracts with unknown/undefined chemical components. Instead, all essential components necessary to support the desired growth/proliferation of desired cell types are chemically defined. Most, if not all, of these individual components can be readily purchased as commercial products from various venders, such as Sigma-Aldrich Corp. (St. Louis, Mo.), GIBCO-Invitrogen Corp. (Carlsbad, Calif.); Calbiochem, and/or BD Biosciences (San Jose, Calif.), etc.

In certain other embodiments, the presence in the subject medium of serum and/or tissue extracts, especially in trace amounts, would not substantially interfere with the characteristics of the medium, such as inhibiting the ability of the subject medium to support long-term undifferentiated cell growth and/or proliferation without a significant decrease in differentiation potential.

The invention also provides normal primary cells produced and/or isolated using the subject methods and media.

The invention also provides a method to carry out a pharmaceutical/biotechnology product discovery and development, comprising of creation of tumor models derived from various normal cell types isolated and propagated using the media and methods of the instant invention, and screening for drug molecule or lead compound libraries in order to identify molecules that target tumor cells but not their normal precursor cells.

The phrases “cell culture medium,” “culture medium” (plural “media” in each case) and “medium formulation” refer to a nutritive solution for cultivating cells and may be used interchangeably.

The cell culture media of the present invention are aqueous-based (but can be reconstituted from dry powder and/or frozen components), comprising a number of ingredients in a solution of, preferably deionized and/or distilled, water.

The term “ingredient” refers to any compound, whether of chemical or biological origin, that can be used in cell culture media to maintain or promote the growth of proliferation of cells. The terms “component,” “nutrient” and ingredient” can be used interchangeably and are all meant to refer to such compounds. Typical ingredients that are used in cell culture media include amino acids, salts, metals, sugars, lipids, nucleic acids, hormones, vitamins, fatty acids, proteins and the like. Other ingredients that promote or maintain cultivation of cells ex vivo can be selected by those of skill in the art, in accordance with the particular need.

By “cell culture” or “culture” is meant the maintenance of cells in an artificial, in vitro environment. It is to be understood, however, that the term “cell culture” is a generic term and may be used to encompass the cultivation not only of individual cells, but also of tissues, organs, organ systems or whole organisms, for which the terms “tissue culture”, “organ culture”, “organ system culture” or “organotypic culture” may occasionally be used interchangeably with the term “cell culture.”

Certain cells, such as human cells must have adequate amounts of 9 amino acids to survive. These so called “essential” amino acids cannot be synthesized from other precursors. However, cysteine can partially meet the need for methionine (they both contain sulfur), and tyrosine can partially substitute for phenylalanine. Such essential amino acids include: Histidine, Isoleucine, Leucine, Lysine, Methionine (and/or cysteine), Phenylalanine (and/or tyrosine), Threonine, Tryptophan, and Valine. In certain embodiments, only Histidine, Isoleucine, Leucine, Lysine, Threonine, Tryptophan, and Valine are included.

Some or all of the ingredients, when admixed together in solution, can form a “basal medium.” To this basal medium, other components, such as at least one nucleotide synthesis and/or salvage pathway precursors (e.g. hypoxanthine), epidermal growth factor (EGF), agents increasing intracellular cyclic adenosine monophosphate (cAMP) levels, and antioxidants, can be added to formulate the complete culture media of the present invention. These latter added components, such as EGF and the cAMP-increasing agent(s) may be added to freshly formulated basal medium, or they may be admixed as in a stock solution stored frozen, preferably at about −20° C. to about −70° C., until being added to basal medium to formulate the complete medium of the present invention.

To the extent that components do not substantially affect the performance of the medium in terms of supporting undifferentiated primary cell growth and proliferation, the subject medium may in certain embodiments include and tolerate the presence of one or more of such components.

One or more components of the medium may also be substituted by other chemicals of similar properties when necessary. Such modified medium without one or more non-essential/unnecessary components are within the scope of the invention. Similarly, a skilled artisan could also determine the optimal level of any given component for a particular cell type, by, for example, testing a range of concentrations (e.g., 10%, 25%, 50%, 75%, 100%, 2-, 5-, 10-, 20-, 50-, 100-, 200-, 500-, 1000-fold higher, or 10%, 25%, 50%, 75%, 100%, 2-, 5-, 10-, 20-, 50-, 100-, 200-, 500-, 1000-fold lower) for each listed component based on or starting from the listed concentration of that particular component. Some components have a listed range of concentrations. The proper or optimal concentration for any particular cell types can also be determined similarly starting from the listed concentration. In doing such tests, initial broad-range concentration tests may be narrowed down later based on the outcomes of the initial experiments. For example, for an initial test, the concentration of one component of interest may be changed to 10⁻³, 10⁻², 10⁻¹, 10-fold, 100-fold, and 1000-fold of the initial concentration. If the 10⁻² test still supports the desired growth, while 10⁻³ fails to, then the 10-fold concentration difference between 10⁻² and 10⁻³ may be further explored in the second round of test to pin-point the best ranges. Thus, media so optimized for specific cell types are also within the scope of the instant invention. As will be readily apparent to one of ordinary skill in the art, the concentration of a given ingredient can be increased or decreased beyond the range disclosed and the effect of the increased or decreased concentration can be determined using only routine experimentation. The optimization of the present media formulations for any specific cell type can be carried out using approaches described by Ham (Ham, Methods for Preparation of Media, Supplements and Substrata for Serum-Free Animal Culture, Alan R. Liss, Inc., New York, pp. 3-21, 1984) and Waymouth (Weymouth, C, Methods for Preparation of Media, Supplements and Substrata for Serum-Free Animal Culture, Alan R. Liss, Inc., New York, pp. 23-68, 1984). The optimal final concentrations for medium ingredients are typically identified either by empirical studies, in single component titration studies, or by interpretation of historical and current scientific literature. In single component titration studies, using animal cells, the concentration of a single medium component is varied while all other constituents and variables are kept constant and the effect of the single component on viability, growth or continued health of the animal cells is measured.

It will be understood that certain vitamins and hormones listed herein can exist in different forms, as known in the art (e.g., different naturally occurring or non-naturally occurring forms), and can be used as substitutes for one another. It will also be appreciated that where the instant application discloses a vitamin or hormone, the invention should be understood to encompass embodiments in which any form of such vitamin or hormone having similar biological activity (or compound(s) that can be modified or metabolized in cell culture medium or intracellularly to provide a biologically active form) is used in the inventive media and/or method(s).

It will be appreciated that compounds such as estrogen, progesterone, thyroid hormone, hydrocortisone, insulin, etc., can be substituted in whole or in part by other compounds (naturally occurring or non-naturally occurring, isolated from natural sources or at least in part chemically synthesized) that are agonists of the estrogen receptor, progesterone receptor, thyroid hormone receptor, glucocorticoid receptor, insulin receptor, respectively. A number of such compounds are known in the art.

The medium ingredients can be dissolved in a liquid carrier or maintained in dry form. If dissolved in a liquid carrier at the preferred concentrations shown above (i.e., a “1× formulation”), the pH of the medium should be adjusted to about 7.0-7.6, for instance about 7.1-7.5, or about 7.2-7.4. The osmolarity of the medium could also be adjusted to about 275-350 mOsm, for instance about 285-325 mOsm, or about 280-310 mOsm. The type of liquid carrier and the method used to dissolve the ingredients into solution vary and can be determined by one of ordinary skill in the art with no more than routine experimentation. Typically, the medium ingredients can be added in any order.

A cell culture medium is composed of a number of ingredients and these ingredients vary from one culture medium to another. A “1× formulation” is meant to refer to any aqueous solution that contains some or all ingredients found in a cell culture medium at working concentrations. The “1× formulation” can refer to, for example, the cell culture medium or to any subgroup of ingredients for that medium. The concentration of an ingredient in a 1× solution is about the same as the concentration of that ingredient found in a cell culture formulation used for maintaining or cultivating cells in vitro. A cell culture medium used for the in vitro cultivation of cells is a 1× formulation by definition. When a number of ingredients are present, each ingredient in a 1× formulation has a concentration about equal to the concentration of those ingredients in a cell culture medium. For example, RPMI-1640 culture medium contains, among other ingredients, 0.2 g/L L-arginine, 0.05 g/L L-asparagine, and 0.02 g/L L-aspartic aced. A “1× formulation” of these amino acids contains about the same concentrations of these ingredients in solution. Thus, when referring to a “1× formulation,” it is intended that each ingredient in solution has the same or about the same concentration as that found in the cell culture medium being described. The concentrations of ingredients in a 1× formulation of cell culture medium are well known to those of ordinary skill in the art. See Methods For Preparation of Media, Supplements and Substrate For Serum-Free Animal Cell Culture Allen R. Liss, N.Y. (1984). The osmolality and/or pH, however, may differ in a 1× formulation compared to the culture medium, particularly when fewer ingredients are contained in the 1× formulation.

A “10× formulation” is meant to refer to a solution wherein each ingredient in that solution is about 10 times more concentrated than the same ingredient in the cell culture medium. For example, a 10× formulation of RPMI-1640 culture medium may contain, among other ingredients, 2.0 g/L L-arginine, 0.5 g/L L-asparagine, and 0.2 g/L L-aspartic acid (compare 1× formulation, above). A “10× formulation” may contain a number of additional ingredients at a concentration about 10 times that found in the 1× culture medium. As will be readily apparent, “25× formulation”, “50× formulation”, “100× formulation, “500× formulation”, and “1000× formulation” designate solutions that contain ingredients at about 25-, 50-, 100-, 500-, or 1000-fold concentrations, respectively, as compared to a 1× cell culture medium. Again, the osmolality and pH of the media formulation and concentrated solution may vary. Preferably, the solutions comprising ingredients are more concentrated than the concentration of the same ingredients in a 1× media formulation. The ingredients can be 10-fold more concentrated (10× formulation), 25-fold more concentrated (25× formulation), 50-fold more concentrated (50× formulation), or 100-fold more concentrated (100× formulation). More highly concentrated formulations can be made, provided that the ingredients remain soluble and stable. See e.g. U.S. Pat. No. 5,474, which is directed to methods of solubilizing culture media components at high concentrations.

If the media ingredients are prepared as separate concentrated solutions, an appropriate (sufficient) amount of each concentrate is combined with a diluent to produce a 1× medium formulation. Typically, the diluent used is water but other solutions including aqueous buffers, aqueous saline solution, or other aqueous solutions may be used according to the invention.

The culture media of the present invention are typically sterilized to prevent unwanted contamination. Sterilization may be accomplished, for example, by filtration through a low protein-binding membrane filter of about 0.1-1.0 μm pore size (available commercially, for example, from Millipore, Bedford, Mass.) after admixing the concentrated ingredients to produce a sterile culture medium. Alternatively, concentrated subgroups of ingredients may be filter-sterilized and stored as sterile solutions. These sterile concentrates can then be mixed under aseptic conditions with a sterile diluent to produce a concentrated 1× sterile medium formulation. Autoclaving or other elevated temperature-based methods of sterilization are not favored, since many of the components of the present culture media are heat labile and will be irreversibly degraded by temperatures such as those achieved during most heat sterilization methods.

Many tissue culture media typically contain one or more antibiotics, which are not necessary for cell growth/proliferation per se, but are present to inhibit the growth of other undesirable microbes, such as bacteria and/or fungi. Antibiotics are natural chemical substances of relatively low molecular weight produced by various species of microorganisms, such as bacteria (including Bacillus species), actinomycetes (including Streptomyces) and fungi that inhibit growth of or destroy other microorganisms. Substances of similar structure and mode of action may be synthesized chemically, or natural compounds may be modified to produce semi-synthetic antibiotics. These biosynthetic and semi-synthetic derivatives are also effective as antibiotics. The major classes of antibiotics are: (1) the β-lactams, including the penicillins, cephalosporins and monobactams; (2) the aminoglycosides, e.g., gentamicin, tobramycin, netilmycin, and amikacin; (3) the tetracyclines; (4) the sulfonamides and trimethoprim; (5) the fluoroquinolones, e.g., ciprofloxacin, norfloxacin, and ofloxacin; (6) vancomycin; (7) the macrolides, which include for example, erythromycin, azithromycin, and clarithromycin; and (8) other antibiotics, e.g., the polymyxins, chloramphenicol and the lincosamides. Antibiotics accomplish their anti-bacterial effect through several mechanisms of action which can be generally grouped as follows: (1) agents acting on the bacterial cell wall such as bacitracin, the cephalosporins, cycloserine, fosfomycin, the penicillins, ristocetin, and vancomycin; (2) agents affecting the cell membrane or exerting a detergent effect, such as colistin, novobiocin and polymyxins; (3) agents affecting cellular mechanisms of replication, information transfer, and protein synthesis by their effects on ribosomes, e.g., the aminoglycosides, the tetracyclines, chloramphenicol, clindamycin, cycloheximide, fucidin, lincomycin, puromycin, rifampicin, other streptomycins, and the macrolide antibiotics such as erythromycin and oleandomycin; (4) agents affecting nucleic acid metabolism, e.g., the fluoroquinolones, actinomycin, ethambutol, 5-fluorocytosine, griseofulvin, rifamycins; and (5) drugs affecting intermediary metabolism, such as the sulfonamides, trimethoprim, and the tuberculostatic agents isoniazid and para-aminosalicylic acid. Some agents may have more than one primary mechanism of action, especially at high concentrations. In addition, secondary changes in the structure or metabolism of the bacterial cell often occur after the primary effect of the antimicrobial drug.

Thus for convenience and other practical reasons, the subject media may be additionally supplemented by one or more antibiotics or other substances that inhibit the growth/proliferation of undesirable bacteria/fungi/virus. In other embodiments, however, the subject medium may be free of any antibiotics to ensure optimum growth of primary cells. Extra care should be taken when handling cells growing in antibiotic-free medium in order to avoid possible contamination.

The medium of the instant invention can be made from individual components separately purchased from various chemical vendors. Alternatively, certain commercial medium may be conveniently mixed and supplemented by additional components for make the subject medium. For example, in some embodiments, the subject medium may comprise about 50% Medium 199 (M 199) and about 50% F12 (Ham) medium, supplemented with B27 (Invitrogen), HEPES, estrogen and β-mercaptoethanol. The invention thus provides methods of making a tissue culture medium comprising supplementing a commercially available cell culture medium or mixture of two or more such media by adding one or more components disclosed herein

The invention provides a tissue culture medium comprising the components sufficient the growth of cells. The composition of these media may be varied. For example, the concentration of any of the components may be independently varied by up to 10%, 20%, 30%, 40%, or 50%, or by up to a factor of up to 2-3 fold, relative to the original concentrations. In one embodiment, the concentrations of each of the components vary by not more than 10% from the listed value. In one embodiment, the concentrations of each of the components vary by not more than 25% from the listed value. Unless otherwise indicated, as used herein, variation by up to X % means variation by ±X % with respect to the listed value. For example, if the listed value is 100 ng/ml, variation by 25% means that the value can range between 75 ng/ml and 125 ng/ml (i.e., 75-125 ng/ml). Unless otherwise indicated, where a range of values is disclosed, endpoints are included within the range. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate to any intervening value or range defined by any two values in the series, where the lowest value may be taken as a minimum and the greatest value may be taken as a maximum. For any embodiment of the invention in which a value is prefaced by the term “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”.

It will be appreciated that certain of the components may be provided as salts, esters, biologically active metabolites or derivatives, or as precursors that are metabolized, processed, or broken down by the cell or in the medium to yield a biologically active form of certain of the components disclosed herein. “Biologically active” in this context refers to the ability of the component to exert its desired effect on a cell when present in a cell culture medium.

The medium of the instant invention may be liquid or solid powder, or a combination of both. The liquid form may be a complete medium, which contains all the components sufficient to support the growth/proliferation of the target cells. Alternatively, the liquid media may be stored as separate packages, such that each individual package may be stored at its appropriate conditions (temperature, humidity, etc.). For example, most of the components, if desired to be in a medium of the instant invention, can be pre-dissolved in a single solution and stored at appropriate conditions (e.g. 4° C. in a dark and dry place, etc.). Other components, which could be unstable at the storage conditions for the other components, or which could react slowly with other components, or which is otherwise better kept as a separate stock, may be stored under a different set of conditions (e.g. −20° C. or −80° C., etc.). It is only shortly or immediately before use are these separately stored components brought together to constitute the whole medium. Each separate package may be marketed or sold separately, or as different concentrated stocks (e.g. 2×, 5×, 10×, 100×, 1000×, etc.). In some embodiments, a medium of the instant invention is marketed or sold together with one or more cell lines (e.g., one or more cell line(s) disclosed herein), for whose culture said medium is suitable.

Similarly, the complete medium or individual components, packages thereof could be in the form of dry powder, which, upon reconstitution with an aqueous solution (such as water), will yield the desired medium, or its concentrated stocks (2×, 5×, or 10×, etc.).

Components that can be, or better kept as separate stocks just prior to use include: growth factors (e.g. Epidermal Growth Factor), hormones (e.g. estrogen, progesterone, testosterone), other unstable enzymes/proteins (e.g. transferrin, insulin, cholera toxin, etc.), steroids (e.g. hydrocortisone, cholesterol), vitamins (Vitamins A, Bi₂, K₃), pH indicators (e.g. phenol red), one or more buffer components (e.g. sodium biocarbonate, HEPES) and other chemicals (e.g. glutathione, 17-β-estradiol, O-phosphoryl ethanolamine, etc.).

In certain embodiments, at least some or all components of the medium is in liquid/aqueous form. In other embodiments, at least some or all components of the medium is in solid/powder form. The media of the invention are suitable for a variety of primary glandular epithelial cells, including epithelial cells from breast (luminal), prostate, lung, GI tract (e.g. salivary gland, small and large intestine, colon, stomach, pancreas, liver, gall bladder, etc.), cervix, endometrium/uterus, and ovary. The media may also be suitable for culturing primary endothelial cells.

The media of the invention are suitable for a variety of primary cells from different mammals, including human and other non-human mammals. The latter further includes: non-human primates (e.g. monkey, gorilla, etc.), mouse, rat, rabbit, domestic cattle, horse, pig, sheep, goat, dog, and cat. In certain embodiments, the medium supports the growth, proliferation, and/or differentiation of cells from normal glandular epithelial cells of an organ/tissue selected from: breast, prostate, ovary, pancreas, stomach, intestine, colon, endocervix, kidney, skin, lung, uterus, parotid gland, or fallopian tube.

Another aspect of the invention relates to the use of the subject medium for isolating primary cells, such as primary mammalian glandular epithelial cells substantially free of other cell types, including stromal cells and myoepithelial cells.

Although not limiting, the method of the invention is particularly suitable to isolate primary glandular epithelial cells, such as mammary glandular epithelial cells. Other primary cell sources include such tissues as: breast, prostate, ovary, pancreas, stomach, intestine, colon, endocervix, kidney, skin, lung, uterus, parotid gland, or fallopian tube.

“Substantially free” as used herein refers to at least about 80% pure, preferably 85%, 90%, 95%, 99% or more pure population of the desired cells in the whole cell population.

In one embodiment, a tissue with the desired glandular epithelial cells (such as breast, prostate, ovary, pancreas, stomach, intestine, colon, endocervix, kidney, skin, lung, uterus, parotid gland, or fallopian tube, etc.) is obtained from a human or non-human patient, and minced down to chunks/cubes/fragments of about 1-2 mm in dimension. Cells in the fragments are then separated by, for example, overnight collagenase digestion in suitable buffer and temperature (e.g. Hanks' buffered saline solution at 37° C. overnight). Other methods, such as mechanical means (passing the minced tissue through a steel mesh using a plunger, optionally followed by a cotton wool column, etc.) may also be used.

In addition to be suitable for culturing normal mammalian primary epithelial cells, the subject media is also suitable for culturing primary cell-derived, tumor stem cell-like cells. Thus, yet another aspect of the invention relates to isolating/establishing/purifying model tumor stem cells, and using such model tumor stem cells for screening agents specifically or preferentially targeting tumor stem cells. The identification of a breast cancer stem cell represents a major step forward in the elucidation of the breast cancer tumor hierarchy and signals the beginning of a new era of breast cancer research. The most important outcome of these studies extends beyond the breast cancer field to cancer research in general. Although the cancer stem cell hypothesis is well established, much modern cancer research still treats tumors as homogeneous collections of cells that can be simply disrupted for biochemistry studies or for gene expression profiling. The focus of future studies in cell signaling, molecular and cellular comparisons of normal and tumorigenic pathways, gene expression profiling, and drug development must include the cancer stem cell. This in turn will require the use of primary tissue and not cell lines, functional transplantation assays for the cancer stem cell, and cell purification using cell surface or metabolic properties to isolate enriched populations

Thus an aspect of the instant invention provides a relatively simple way to isolate, purify, enrich, and/or establish cancer stem cell-like cells derived from normal primary cells of the invention, including those derived from primary breast epithelial cells, by maintaining and expanding said cells in a medium according to the invention. As such it allows direct comparison of normal primary cells to tumorigenic and metastatic cancer stem cells that are directly derived from these normal cells. Such a direct comparison would not be possible for tumor stem cells that are isolated from patient samples, as the tumor initiating normal cells have been already transformed and no longer exist by definition.

The invention also provides an in vitro method of identifying an agent which enhances or positively affects one or more characteristics of tumorigenic cells, the characteristics including: differentiation, apoptosis, sensitivity to chemotherapy/radiotherapy, or senescence, the method comprising: (1) contacting a cell culture of tumorigenic cells in the medium of invention with a candidate agent to be assessed for its ability to enhance or positively affect the one or more characteristics of the tumorigenic cells, under conditions appropriate for the agent to enter cells; (2) determining the extent to which the characteristics is enhanced or positively affected in the presence of the candidate agent to be assessed; and, (3) comparing the extent determined with the characteristics of the tumorigenic cells under the same conditions, but in the absence of the candidate agent to be assessed, wherein if the characteristics is substantially enhanced or positively affected in the presence of the candidate agent to be assessed than in its absence, the candidate agent to be assessed is an agent which enhances or positively affects one or more the characteristics of the tumorigenic cells.

The invention also provides an in vivo method of identifying an agent which inhibits or negatively affects one or more characteristics of tumor generated by tumorigenic cells in a tumor model, the characteristics including: tumor stem cell frequency, tumor growth, tumor differentiation invasiveness, metastasis, or angiogenesis, the method comprising: (1) introducing to test animals tumorigenic cells which have been expanded in the medium of invention to generate tumors; (2) administering a candidate agent to the test animals to assess its ability to inhibit or negatively affect the one or more characteristics of the tumor; and, (3) determining the extent to which the characteristics is inhibited or negatively affected in the presence of the candidate agent; wherein if the characteristics is substantially inhibited or negatively affected in the presence of the candidate agent to be assessed than in its absence, the candidate agent to be assessed is an agent which inhibits or negatively affects one or more the characteristics of the tumor. Another aspect of the invention provides an in vivo method of determining the effect of at least two candidate agents which potentially affect one or more characteristics of tumor generated by tumorigenic cells in a tumor model, the characteristics including: tumor growth, invasiveness, metastasis, or angiogenesis, the method comprising: (a) associating each candidate agent with a unique detectable marker, wherein presence of the detectable marker substantially matches the presence of the candidate agent; (b) dividing tumorigenic cells expanded in the medium of the invention into separate groups according to the number of candidate agents to be tested; (c) contacting one group of tumorigenic cells with one candidate agent; (d) introducing to test animals tumorigenic cells of step (c) to generate tumors; (e) determining the extent to which the one or more characteristics is affected in the presence of the candidate agent; (f) determining the presence of detectable markers; wherein the presence of a candidate agent is determined by the presence of its associated detectable marker, and wherein if the one or more characteristics is substantially affected in the presence of the candidate agent than in its absence, the candidate agent is an agent which affects one or more the characteristics of the tumor. In certain embodiments, each group of tumorigenic cells comprises 100 or less tumorigenic cells. In certain embodiments, the detectable marker is a DNA bar code. In certain embodiments, the detectable marker is a fluorescent marker. In certain embodiments, the agent is an RNAi molecule. In certain embodiments, the agent is a siRNA molecule. In certain embodiments, the agent is a chemical compound.

In a typical mouse xenograft experiment 1 to 10 million cells are injected into an animal model (such as mouse) in order to form a tumor. It has however been shown that a system similar to ours allows tumor formation using as low as 10 cells (Ince et al., 2007 Cancer Cell, 12(2):160-170). Therefore, instead of the traditional one reagent (or one condition) per animal model, our system can screen a plurality of reagents or conditions a single animal model. For example, each batch, comprising about 10-100 cells, may be treated with a biological agent or a chemical agent. Exemplary biological agents can be an RNAi molecule, an siRNA construct, or a genetic inhibitor that targets a particular gene; exemplary chemical agent may be a small molecule compound. Each candidate agent should be associated with a unique detectable marker (such as a DNA bar code), so that the presence of the detectable marker substantially matches the presence of the candidate agent. Multiple batches of cells, each treated with a different candidate agent, can then be pooled and injected into a single animal model. Thus multiple candidate agents may be screened in a single xenograft experiment, greatly increasing the efficiency of drug screening and making in vivo high throughput screening possible. Techniques of DNA bar code tagging of chemical libraries is known in the art. The construction of libraries of compounds which carry a unique DNA sequence as a “bar-code” for each library member can facilitate the identification of binding molecules and to speed-up and miniaturize screening procedures. In principle, very large libraries of compounds can be panned with the target protein of interest immobilized on a suitable support, followed by the identification of the preferentially enriched compounds by procedures which may involve PCR amplification of the DNA codes, sequencing, and/or hybridization to a DNA microarray. DNA-encoded libraries of small organic molecules also facilitate the construction of large, encoded self-assembling chemical libraries for the identification of high-affinity binders to protein targets. For example, Dumelin et al. (Bioconjugate Chem., 17 (2), 366-370, 2006) describes the construction a library of 477 chemical compounds, coupled to 48mer-oligonucleotides, each containing a unique six-base sequence serving as “bar-code” for the identification of the chemical moiety. The functionality of the library was confirmed by selection and amplification of both high- and low-affinity binding molecules specific to streptavidin. Gartner et al. (2004 Sep. 10; 305(5690): 1601-5) describes using multistep DNA-templated organic synthesis to translate libraries of DNA sequences, each containing three “codons,” into libraries of sequence-programmed synthetic small-molecule macrocycles. The resulting DNA-macrocycle conjugates were subjected to in vitro selections for protein affinity. The identity of a single macrocycle possessing known target protein affinity was inferred through the sequence of the amplified DNA template surviving the selection. Brummelkamp et al. (Nature Chemical Biology 2, 202-206 (2006) describes the application of a large-scale RNA interference-based short hairpin RNA (shRNA) barcode screen to gain insight in the mechanism of action of nutlin-3. Noren and Noren (Methods. 2001 February; 23(2): 169-78) teaches a method for construction of high-complexity (>10⁹ independent clones) random peptide libraries. Scheuermann et al. (J Biotechnol. 2006 Dec. 1; 126(4):568-81. Epub 2006 Jun. 9) summarizes various techniques of DNA-encoded chemical libraries. Takahashi et al. (Trends Biochem Sci. 2003 March; 28(3): 159-65) describes libraries in which mRNA molecules are covalently attached to the peptide or protein they encode. These mRNA-protein fusions enable in vitro selection of peptide and protein libraries of >10¹³ different sequences. The invention also provides a method of identifying a gene whose expression is related to/involved in metastasis of such cells in vivo, comprising: (1) introducing a candidate gene into tumorigenic cells in a medium of the subject invention, thereby producing modified tumorigenic cells; (2) introducing the modified tumorigenic cells to test animals; (3) maintaining the test animals under conditions appropriate for formation of tumors and metastasis to occur; and (4) determining whether metastasis of the modified tumorigenic cells occurs, wherein, if metastasis occurs, the candidate gene is a gene whose expression in a tumorigenic cell is related to/involved in metastasis of such cells in vivo.

The invention also provides a method of identifying a gene whose expression in a tumorigenic cell is related to/involved in invasion of such cells in vivo, comprising: (1) introducing a candidate gene into tumorigenic cells in a medium of the subject invention, thereby producing modified tumorigenic cells; (2) introducing the modified tumorigenic cells to test animals; (3) maintaining the test animals under conditions appropriate for formation of tumors and invasion to occur; and (4) determining whether invasion of the modified tumorigenic cells occurs, wherein, if invasion occurs, the candidate gene is a gene whose expression in a tumorigenic cell is related to/involved in invasion of such cells in vivo.

The invention also provides a method of identifying a gene product which is expressed in tumor cells but not in normal or non-tumorigenic cells of the same type, or a gene product which is not expressed in tumor cells but is expressed in normal or non-tumorigenic cells of the same type, comprising: (1) analyzing tumorigenic cells cultured in a medium of the invention; (2) analyzing normal parental cells of which the tumorigenic cells are a variant for gene products; and, (3) comparing gene products produced by the tumorigenic cells and the normal parental cells, whereby a gene product which is expressed in tumorigenic cells but not in normal parental cells, or a gene product which is not expressed in tumorigenic cells but is expressed in normal parental cells is identified, thereby identifying a gene product which is expressed in tumor cells but not in normal cells of the same type, or a gene product which is not expressed in tumorigenic cells but is expressed in normal cells of the same type.

The invention also contemplates equivalent embodiments in the cancer stem cells, all genetically and otherwise modified derivatives, and metastatic derivatives isolated/purified using the methods and media of the instant invention.

One embodiment of the invention provides methods to isolate mammary cells. One source of mammary epithelial cells comes from milk. Early lactation and post-weaning milks give the highest yield of epithelial cells. In a typical protocol for milk collection, 2-7 days postpartum milk (about 5-20 mL per patient) is collected by expressing milks manually into a sterile container. The milks are pooled and diluted 1:1 with RPMI 1640 medium to facilitate centrifugation at 600-1000 g for about 20 minutes. The supernatant is carefully removed without disrupting the cell pellet, which is then washed 2-4 times with RPMI 1640 with 5% FCS until supernatant is not turbid. Resuspend the packed cell volume in growth medium and plate 50 μL packed cells in 5-cm dishes in 6 mL the subject (primary) growth medium. Incubate at 37° C. in 5% CO₂. Follow the rest of the subject methods to isolate/subculture glandular epithelial cells.

In an alternative embodiment, mammary epithelial cells can be isolated from reduction mammoplasty tissue, or other surgical procedures. An enzymatic dissociation technique, (modified from Hallowes et al, Cancer Res. 37: 2492-2505, 1977) coupled with crude dissection, yields large amount of pure epithelial tissues from each individual donor.

Human mammary tissue can be obtained as discarded tissues from mammoplasty surgery. Such material (preferably freshly dissected within 1-2 hours, no more than 5 hours) is placed in sterile containers with sterile buffer or Ham's F-12 medium with insulin, antibiotics, and 10% FBS (e.g. 10 μg/mL insulin, 100 U/mL penicillin, 100 μg/mL streptomycin, 50 U/mL polymixin B, and 5 μg/mL fungizone in Ham's F12 medium). If not used immediately as above, tissues can be stored at 4° C. for up to 72 hours.

Transfer cut pieces of the tissue into a Petri dish. Separate the epithelial areas (appear as embedded white strands) from the stromal matrix and the grossly fatty material in sterile 150 mm Petri dishes using a combination of sterile scalpel, forceps, and scissors. Then transfer the minced epithelium-containing tissue into a conical centrifuge tube (50 or 15 mL) with the tissue making up no greater than ⅓ of the volume of the tube. Bring the tube to full volume with tissue digesting medium (final concentration of 150 U/mL collagenase, 100 U/mL hyaluronidase, and 10% FBS in tissue mix medium), leaving only a small air space to allow for gentle mixing during rotation overnight at 37° C. Centrifuge the tubes at 600 g for 5 minutes. Discard the supernatant fat and medium. Optionally, dilute a small volume of the pellet in medium to check for degree of digestion. Digestion is complete when microscopic examination shows clumps of cells (organoids) with ductal, alveolar, or ductal-alveolar structures free from attached stroma. If digestion is incomplete, repeat the digestion for another 4-12 hours until completion. When digestion is complete, centrifuge tubes at 600 g for 5 minutes, carefully aspirate supernatant, and resuspend pellet in tissue mix medium. Optionally, filter the mixture, a few milliliters a time, through a sterile 150 μL pore-size filter, and wash the organoids left on the filter a few times with 2-3 mL of medium. Flip the filter and wash the organoids off into a sterile container to collect the 150 μL organoid pool, which contains mostly ductal structures. The filtering step is often not necessary in many cases.

Repeat the same procedure using 95 μL pore-size filter to collect 95 μL organoid pool, which contains mostly smaller ductal and alveolar structures. The filtrate contains mostly small epithelial clumps and stromal cells. Transfer the 150 μL and the 95 μL organoids collections, and the final filtrate to 50-mL tubes and centrifuge at 600 g for 5 minutes. Aspirate the supernatant; resuspend the pellets in each tube by adding 1 mL medium for each 0.1 mL of packed pellet. Transfer the resuspended materials, drop by drop, onto culture surfaces to cover different areas of the dish. Then add the subject primary medium and follow the rest of the protocol.

In yet another embodiment, the tissues are minced down to fragments about 1-2 mm in size, and digested with collagenase in Hank's buffered saline solution (HBSS) at 37° C. overnight. The resulting mixture of organoids that contain organoids which are separated from single cells that are mostly stromal and myoepithelial by three consecutive rounds of centrifugation (5 min. at 200×g, 100×g and 20×g). The pellets and supernatants are collected in six fractions (200 g, 100 g and 20 g) and filtered through a 75 μm mesh, and subsequently a 45 μm mesh. All filtered fractions are plated in Primaria™ tissue culture dishes (or similar mixed-charged surfaces) in the subject culture medium. The cells are grown for 7 days during which medium is changed every day. On day 8 and 9 plates are trypsinized with 0.025% trypsin removing all stromal cells. Epithelial cells are harvested with 0.15% trypsin and transferred to new Primaria™ plates in the subject medium. After one week, cells are transferred to the medium of the invention and subcultured in the same medium.

In some embodiments, the cells are plated at a density of at least 0.5×10³ cells/cm² up to about 10⁶ cells/cm². In some embodiments the cells are plated at a density of at least 10⁴ cells/cm″, e.g., between 1-2×10⁴ cells/cm² or between 1-5×10⁴ cells/cm² In some embodiments the cells are plated at between 0.5×10³ cell/cm² and 5×10⁵ cells/cm². It will be appreciated that optimal density may vary for different cell types. The tissue culture medium may be changed at varying intervals, e.g., at intervals of between 12 and 72 hours. In some embodiments, the tissue culture medium is changed relatively frequently, e.g., every 12-24 hours. In some embodiments the tissue culture medium is changed at longer intervals, e.g., every 24-72 hours. The cells may be transferred or passaged at varying intervals, e.g., between 3 days to 4 weeks, e.g., on average once a week, once every two weeks, etc. The afore-mentioned embodiments are exemplary and are not intended to limit the invention. In a specific embodiment, small organoids from breast tissues, rather than larger ones, are used as cell sources, since they tend to yield better results than larger ones. While not wishing to be bound by any particular theory, it is possible that smaller organoids come from smaller lobules of the breast tissues, from where about 99% of breast cancers originate, instead of from larger ducts. It is also possible that the smaller organoids from small lobules are biologically distinct from the larger organoids from large ducts.

Fibroblast cell growth is generally not a problem since the subject medium does not support fibroblast cell growth effectively. However, if fibroblast growth is observed, especially in the 150 μL and 95 μL organoid fraction, fibroblasts can be removed by differential trypsinization as follows. When the epithelial patches are large, aspirate medium, wash once with saline-typsin-versene (SW), and then add 0.5 mL SW per 60-mm dish. Leave SW on cells at room temperature for about 1-2 minutes, with continued observation under the microscope. Knock the dish gently. When the fibroblasts are observed to detach while the epithelial cells remain adherent, remove the SW. Wash cells 2-3 times with sterile PBS and refeed with fresh growth medium.

The isolated mammary epithelial cells can be further characterized by immuno-staining using marker protein antibodies which hare well-known in the art. This may be necessary since the breast is a complex tissue with many different cell types whose lineages are not well defined. For example, fibroblast from stromal tissues may be present in the culture. In addition, one or both of the major breast epithelial cells, luminal and basal epithelial cells, could be proliferating in the culture. All these could be further complicated by phenotypic modulation that occurs in culture. Fortunately, many monoclonal antibodies against various immunological markers have been development over the years to at least partially solve these problems. Such markers can be used not only to define specific phenotypes in vivo, but also to identify phenotypes of cultured cells. Among them, the expression profiles of various epithelial keratins have been extremely useful, since the expression profiles are maintained in culture as compared to in vivo expression profiles.

Specifically, all luminal epithelial cells express keratin 8 and 18 and most express 19, whereas all the basal cells express keratins 5 and 14, and do not express keratins 8 and 18. Keratin 7 is expressed in both cell types throughout the gland and keratin 19 and 14 are also expressed by both cell types in the large ducts, but not in the TDLU (terminal ductal lobular units). Other antibodies are antibodies directed to a polymorphic epithelial mucin (PEM) that is expressed by luminal epithelial cells (Burchell et ai, 1983; Gendler et al., 1988), to smooth muscle actin, and to CALLA (common leukocytic leukemia antigen), which is specifically expressed by myoepithelial cells.

There are at least 17 histological types of human breast cancers, the majority being invasive ductal carcinoma (about 80%), followed by invasive lobular carcinoma (about 10-15%). Histologically, the squamous type represents less than 1% of the human breast cancer.

The instant invention provides a medium for isolating and culturing mammary progenitor cells which can grow long-term in the subject chemically-defined medium, and subsequently, under appropriate conditions, differentiate.

The subject medium supports growth of isolated mammary progenitor cells in culture for at least about 4 weeks, 6 weeks, 10 weeks, 12 weeks, 14 weeks, 15 weeks or more without reaching senescence. This corresponds to at least about 15, 20, 25, 30, 35 or more population doubling (PD) without reaching senescence. Similarly isolated cells growing in other media typically reach senescence after about 3 weeks, or 3-15 weeks PD in culture.

The medium of the invention is also well suited for the culture of adipocyte precursors (APC) and mesenchymal stem cells (MSC). MSC cultured in the medium of the invention maintain their differentiation potential and can terminally differentiate into adipocytes and osteocytes respectively when adipogenic or osteogenic factors are added to the medium.

In the present invention, “isolated” refers to material removed from its original environment (e.g., the natural environment if it is naturally occurring), and thus is altered “by the hand of man” from its natural state. For example, an isolated polynucleotide could be part of a vector or a composition of matter, or could be contained within a cell, and still be “isolated” because that vector, composition of matter, or particular cell is not the original environment of the polynucleotide. The term “isolated” does not refer to genomic or cDNA libraries, whole cell total or mRNA preparations, genomic DNA preparations (including those separated by electrophoresis and transferred onto blots), sheared whole cell genomic DNA preparations or other compositions where the art demonstrates no distinguishing features of the polynucleotide/sequences of the present invention. Further examples of isolated DNA molecules include recombinant DNA molecules maintained in heterologous host cells or purified (partially or substantially) DNA molecules in solution. Isolated RNA molecules include in vivo or in vitro RNA transcripts of the DNA molecules of the present invention. However, a nucleic acid contained in a clone that is a member of a library (e.g., a genomic or cDNA library) that has not been isolated from other members of the library (e.g., in the form of a homogeneous solution containing the clone and other members of the library) or a chromosome removed from a cell or a cell lysate (e.g., a “chromosome spread”, as in a karyotype), or a preparation of randomly sheared genomic DNA or a preparation of genomic DNA cut with one or more restriction enzymes is not “isolated” for the purposes of this invention. As discussed further herein, isolated nucleic acid molecules according to the present invention may be produced naturally, recombinantly, or synthetically. In the present invention, a “secreted” protein refers to a protein capable of being directed to the ER, secretory vesicles, or the extracellular space as a result of a signal sequence, as well as a protein released into the extracellular space without necessarily containing a signal sequence. If the secreted protein is released into the extracellular space, the secreted protein can undergo extracellular processing to produce a “mature” protein. Release into the extracellular space can occur by many mechanisms, including exocytosis and proteolytic cleavage.

“Polynucleotides” can be composed of single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, polynucleotides can be composed of triple-stranded regions comprising RNA or DNA or both RNA and DNA. Polynucleotides may also contain one or more modified bases or DNA or RNA backbones modified for stability or for other reasons. “Modified” bases include, for example, tritylated bases and unusual bases such as inosine. A variety of modifications can be made to DNA and RNA; thus, “polynucleotide” embraces chemically, enzymatically, or metabolically modified forms. The expression “polynucleotide encoding a polypeptide” encompasses a polynucleotide which includes only coding sequence for the polypeptide as well as a polynucleotide which includes additional coding and/or non-coding sequence.

“Stringent hybridization conditions” refers to an overnight incubation at 42 degree C. in a solution comprising 50% formamide, 5×SSC (750 mM NaCl, 75 mM trisodium citrate), 50 mM sodium phosphate (pH 7.6), 5×Denhardt's solution, 10% dextran sulfate, and 20 μg/ml denatured, sheared salmon sperm DNA, followed by washing the filters in 0.1×SSC at about 50 degree C. Changes in the stringency of hybridization and signal detection are primarily accomplished through the manipulation of formamide concentration (lower percentages of formamide result in lowered stringency); salt conditions, or temperature. For example, moderately high stringency conditions include an overnight incubation at 37 degree C. in a solution comprising 6×SSPE (20×SSPE=3M NaCl; 0.2M NaH2PO4; 0.02M EDTA, pH 7.4), 0.5% SDS, 30% formamide, 100 μg/ml salmon sperm blocking DNA; followed by washes at 50 degree C. with 1×SSPE, 0.1% SDS. In addition, to achieve even lower stringency, washes performed following stringent hybridization can be done at higher salt concentrations (e.g. 5×SSC). Variations in the above conditions may be accomplished through the inclusion and/or substitution of alternate blocking reagents used to suppress background in hybridization experiments. Typical blocking reagents include Denhardt's reagent, BLOTTO, heparin, denatured salmon sperm DNA, and commercially available proprietary formulations. The inclusion of specific blocking reagents may require modification of the hybridization conditions described above, due to problems with compatibility.

The terms “fragment,” “derivative” and “analog” when referring to polypeptides means polypeptides which either retain substantially the same biological function or activity as such polypeptides. An analog includes a proprotein which can be activated by cleavage of the proprotein portion to produce an active mature polypeptide.

The term “gene” means the segment of DNA involved in producing a polypeptide chain; it includes regions preceding and following the coding region “leader and trailer” as well as intervening sequences (introns) between individual coding segments (exons).

Polypeptides can be composed of amino acids joined to each other by peptide bonds or modified peptide bonds, i.e., peptide isosteres, and may contain amino acids other than the 20 gene-encoded amino acids. The polypeptides may be modified by either natural processes, such as posttranslational processing, or by chemical modification techniques which are well known in the art. Such modifications are well described in basic texts and in more detailed monographs, as well as in a voluminous research literature. Modifications can occur anywhere in the polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications. Polypeptides may be branched, for example, as a result of ubiquitination, and they may be cyclic, with or without branching. Cyclic, branched, and branched cyclic polypeptides may result from posttranslation natural processes or may be made by synthetic methods. Modifications include, but are not limited to, acetylation, acylation, biotinylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-linking, cyclization, derivatization by known protecting/blocking groups, disulfide bond formation, demethylation, formation of covalent cross-links, formation of cysteine, formation of pyroglutamate, formylation, gamma-carboxylation, glycosylation, GPI anchor formation, hydroxylation, iodination, linkage to an antibody molecule or other cellular ligand, methylation, myristoylation, oxidation, pegylation, proteolytic processing (e.g., cleavage), phosphorylation, prenylation, racemization, selenoylation, sulfation, transfer-RNA mediated addition of amino acids to proteins such as arginylation, and ubiquitination. (See, for instance, PROTEINS-STRUCTURE AND MOLECULAR PROPERTIES, 2nd Ed., T. E. Creighton, W. H. Freeman and Company, New York (1993); POSTTRANSLATIONAL COVALENT MODIFICATION OF PROTEINS, B. C. Johnson, Ed., Academic Press, New York, pgs. 1-12 (1983); Seifter et al., Meth Enzymol 182:626-646 (1990); Rattan et al., Ann NY Acad Sci 663:48-62 (1992).)

A polypeptide fragment “having biological activity” refers to polypeptides exhibiting activity similar, but not necessarily identical to, an activity of the original polypeptide, including mature forms, as measured in a particular biological assay, with or without dose dependency. In the case where dose dependency does exist, it need not be identical to that of the polypeptide, but rather substantially similar to the dose-dependence in a given activity as compared to the original polypeptide (i.e., the candidate polypeptide will exhibit greater activity or not more than about 25-fold less and, in some embodiments, not more than about tenfold less activity, or not more than about three-fold less activity relative to the original polypeptide.)

Species homologs may be isolated and identified by making suitable probes or primers from the sequences provided herein and screening a suitable nucleic acid source for the desired homologue. “Variant” refers to a polynucleotide or polypeptide differing from the original polynucleotide or polypeptide, but retaining essential properties thereof. Generally, variants are overall closely similar, and, in many regions, identical to the original polynucleotide or polypeptide.

As a practical matter, whether any particular nucleic acid molecule or polypeptide is at least 80%, 85%, 90%, 92%, 95%, 96%, 97%, 98%, 99%, or 100% identical to a nucleotide sequence of the present invention can be determined conventionally using known computer programs. A preferred method for determining the best overall match between a query sequence (a sequence of the present invention) and a subject sequence, also referred to as a global sequence alignment, can be determined using the FASTDB computer program based on the algorithm of Brutlag et al. (Comp. App. Blosci. (1990) 6:237-245). In a sequence alignment the query and subject sequences are both DNA sequences. An RNA sequence can be compared by converting U's to T's. The result of said global sequence alignment is in percent identity. Preferred parameters used in a FASTDB alignment of DNA sequences to calculate percent identity are: Matrix=Unitary, k-tuple=4, Mismatch Penalty-1, Joining Penalty—30, Randomization Group Length=0, Cutoff Score=1, Gap Penalty—5, Gap Size Penalty 0.05, Window Size=500 or the length of the subject nucleotide sequence, whichever is shorter. If the subject sequence is shorter than the query sequence because of 5′ or 3′ deletions, not because of internal deletions, a manual correction must be made to the results. This is because the FASTDB program does not account for 5′ and 3′ truncations of the subject sequence when calculating percent identity. For subject sequences truncated at the 5′ or 3′ ends, relative to the query sequence, the percent identity is corrected by calculating the number of bases of the query sequence that are 5′ and 3′ of the subject sequence, which are not matched/aligned, as a percent of the total bases of the query sequence. Whether a nucleotide is matched/aligned is determined by results of the FASTDB sequence alignment. This percentage is then subtracted from the percent identity, calculated by the above FASTDB program using the specified parameters, to arrive at a final percent identity score. This corrected score is what is used for the purposes of the present invention. Only bases outside the 5′ and 3′ bases of the subject sequence, as displayed by the FASTDB alignment, which are not matched/aligned with the query sequence, are calculated for the purposes of manually adjusting the percent identity score. For example, a 90 base subject sequence is aligned to a 100 base query sequence to determine percent identity. The deletions occur at the 5′ end of the subject sequence and therefore, the FASTDB alignment does not show a matched/alignment of the first 10 bases at 5′ end. The 10 impaired bases represent 10% of the sequence (number of bases at the 5′ and 3′ ends not matched/total number of bases in the query sequence) so 10% is subtracted from the percent identity score calculated by the FASTDB program. If the remaining 90 bases were perfectly matched the final percent identity would be 90%. In another example, a 90 base subject sequence is compared with a 100 base query sequence. This time the deletions are internal deletions so that there are no bases on the 5′ or 3′ of the subject sequence which are not matched/aligned with the query. In this case the percent identity calculated by FASTDB is not manually corrected. Once again, only bases 5′ and 3′ of the subject sequence which are not matched/aligned with the query sequence are manually corrected for.

By a polypeptide having an amino acid sequence at least, for example, 95% “identical” to a query amino acid sequence of the present invention, it is intended that the amino acid sequence of the subject polypeptide is identical to the query sequence except that the subject polypeptide sequence may include up to five amino acid alterations per each 100 amino acids of the query amino acid sequence. In other words, to obtain a polypeptide having an amino acid sequence at least 95% identical to a query amino acid sequence, up to 5% of the amino acid residues in the subject sequence may be inserted, deleted, or substituted with another amino acid. These alterations of the reference sequence may occur at the amino or carboxy terminal positions of the reference amino acid sequence or anywhere between those terminal positions, interspersed either individually among residues in the reference sequence or in one or more contiguous groups within the reference sequence.

As a practical matter, whether any particular polypeptide is at least 80%, 85%, 90%, 92%, 95%, 96%, 97%, 98%, 99%, or 100% identical to, for instance, the amino acid sequences shown in a sequence or to the amino acid sequence encoded by deposited DNA clone can be determined conventionally using known computer programs. A preferred method for determining, the best overall match between a query sequence (a sequence of the present invention) and a subject sequence, also referred to as a global sequence alignment, can be determined using the FASTDB computer program based on the algorithm of Brutlag et al. (Comp. App. Biosci. (1990) 6:237-245). In a sequence alignment the query and subject sequences are either both nucleotide sequences or both amino acid sequences. The result of said global sequence alignment is in percent identity. Preferred parameters used in a FASTDB amino acid alignment are: Matrix=PAM 0, k-tuple=2, Mismatch Penalty—I, Joining Penalty=20, Randomization Group Length=0, Cutoff Score=1, Window Size=sequence length, Gap Penalty—5, Gap Size Penalty—0.05, Window Size=500 or the length of the subject amino acid sequence, whichever is shorter. If the subject sequence is shorter than the query sequence due to N- or C-terminal deletions, not because of internal deletions, a manual correction must be made to the results. This is because the FASTDB program does not account for N- and C-terminal truncations of the subject sequence when calculating global percent identity. For subject sequences truncated at the N- and C-termini, relative to the query sequence, the percent identity is corrected by calculating the number of residues of the query sequence that are N- and C-terminal of the subject sequence, which are not matched/aligned with a corresponding subject residue, as a percent of the total bases of the query sequence. Whether a residue is matched/aligned is determined by results of the FASTDB sequence alignment. This percentage is then subtracted from the percent identity, calculated by the above FASTDB program using the specified parameters, to arrive at a final percent identity score. This final percent identity score is what is used for the purposes of the present invention. Only residues to the N- and C-termini of the subject sequence, which are not matched/aligned with the query sequence, are considered for the purposes of manually adjusting the percent identity score. That is, only query residue positions outside the farthest N- and C-terminal residues of the subject sequence. Only residue positions outside the N- and C-terminal ends of the subject sequence, as displayed in the FASTDB alignment, which are not matched/aligned with the query sequence are manually corrected for. No other manual corrections are to be made for the purposes of the present invention.

Naturally occurring protein variants are called “allelic variants,” and refer to one of several alternate forms of a gene occupying a given locus on a chromosome of an organism. (Genes 11, Lewin, B., ed., John Wiley & Sons, New York (1985).) These allelic variants can vary at either the polynucleotide and/or polypeptide level. Alternatively, non-naturally occurring variants may be produced by mutagenesis techniques or by direct synthesis.

Using known methods of protein engineering and recombinant DNA technology, variants may be generated to improve or alter the characteristics of polypeptides. For instance, one or more amino acids can be deleted from the N-terminus or C-terminus of a secreted protein without substantial loss of biological function. The authors of Ron et al., J. Biol. Chem. 268: 2984-2988 (1993), reported variant KGF proteins having hepanin binding activity even after deleting 3, 8, or 27 amino-terminal amino acid residues. Similarly, Interferon gamma exhibited up to ten times higher activity after deleting 8-10 amino acid residues from the carboxy terminus of this protein (Dobeli et al., J. Biotechnology 7:199-216 (1988)). Moreover, ample evidence demonstrates that variants often retain a biological activity similar to that of the naturally occurring protein. For example, Gayle and co-workers (J. Biol. Chem. 268:22105-22111 (1993)) conducted extensive mutational analysis of human cytokine IL-1a. They used random mutagenesis to generate over 3,500 individual IL-1a mutants that averaged 2.5 amino acid changes per variant over the entire length of the molecule. Multiple mutations were examined at every possible amino acid position. The investigators found that “[most of the molecule could be altered with little effect on either [binding or biological activity].” (See, Abstract.) In fact, only 23 unique amino acid sequences, out of more than 3,500 nucleotide sequences examined, produced a protein that significantly differed in activity from wild-type. Furthermore, even if deleting one or more amino acids from the N-terminus or C-terminus of a polypeptide results in modification or loss of one or more biological functions, other biological activities may still be retained. For example, the ability of a deletion variant to induce and/or to bind antibodies which recognize the secreted form will likely be retained when less than the majority of the residues of the secreted form are removed from the N-terminus or C-terminus. Whether a particular polypeptide lacking N- or C-terminal residues of a protein retains such immunogenic activities can readily be determined by routine methods described herein and otherwise known in the art.

By “biological sample” is intended any biological sample obtained from an individual, body fluid, cell line, tissue culture, or other source which contains the polypeptide of the present invention or mRNA. As indicated, biological samples include body fluids (such as semen, lymph, sera, plasma, urine, synovial fluid and spinal fluid) which contain the polypeptide of the present invention, and other tissue sources found to express the polypeptide of the present invention. Methods for obtaining tissue biopsies and body fluids from mammals are well known in the art. Where the biological sample is to include mRNA, a tissue biopsy is the preferred source.

“Matrigel” is the trade name for a gelatinous protein mixture secreted by mouse tumor cells and marketed by BD Biosciences. This mixture resembles the complex extracellular environment found in many tissues and is used by cell biologists as a substrate for cell culture. A common laboratory procedure is to dispense small volumes of chilled (4° C.) Matrigel onto plastic tissue culture labware. When incubated at 37° C. (body temperature) the Matrigel proteins self-assemble producing a thin film that covers the surface of the labware. Cells cultured on Matrigel demonstrate complex cellular behavior that is otherwise impossible to observe under laboratory conditions. For example, endothelial cells create intricate spiderweb-like networks on Matrigel coated surfaces but not on plastic surfaces. Such networks are highly suggestive of the microvascular capillary systems that suffuse living tissues with blood. Hence, the process by which endothelial cells construct such networks is of great interest to biological researchers and Matrigel allows them to observe this. In some instances, researchers use greater volumes of Matrigel to produce thick three-dimensional gels. The utility of thick gels is that they induce cells to migrate from the surface to the interior of the gel. This migratory behavior is studied by researchers as a model of tumor cell metastasis. Pharmaceutical scientists use Matrigel to screen drug molecules. A typical experiment consists of adding a test molecule to Matrigel and observing cellular behavior. Test molecules that promote endothelial cell network formation are candidates for tissue regeneration therapies whereas test molecules that inhibit endothelial cell network formation are candidates for anti-cancer therapies. Likewise, test molecules that inhibit tumor cell migration may also have potential as anti-cancer drugs. The ability of Matrigel to stimulate complex cell behavior is a consequence of its heterogeneous composition. The chief components of Matrigel are structural proteins such as laminin and collagen which present cultured cells with the adhesive peptide sequences that they would encounter in their natural environment. Also present are growth factors that promote differentiation and proliferation of many cell types. Matrigel contains numerous other proteins in small amounts and its exact composition is unknown. Matrigel is also used as an attachment substrate in embryonic stem cell culture. When embryonic stem cells are grown in the absence of feeder cells, extracellular matrix components such as Matrigel are necessary to maintain the pluripotent, undifferentiated state (self-renewal).

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the present specification, including definitions, will control. In addition, the materials, methods, and examples are illustrative only and not intended to be limiting. Moreover, it is to be noted that despite that fact that the above description concentrates on medical uses, the methods and agents of the invention are also suitable for any non-medical use.

### Examples

### Cell Culture

Approval for culture of reduction mammoplasty tissue was granted by the ethic commission beider Basel (EKBB) and the Emile Muller Hospital in Mulhouse and patients gave informed consent prior to surgery. The patients were healthy women with no previous history of breast cancer. Primary human mammary epithelial cells (PHMECs) were prepared by standard techniques (Stingl et al., 2005, Methods Mol Biol 290, 249-263). Mesenchymal stem cells (MSC) were prepared from breast tissue circumjacent fat tissue according to a standard method (Park et al., 2008, Cell Prolif 41, 859-874). For medium comparison assays, PHMECs were either cultured in MEGM (Dontu et al., 2003, Genes Dev 17, 1253-1270), in HMM (Duss et al., 2007, Breast Cancer Res 9, R38), or in the medium specified in Table II above, over four passages (28 days) prior to fixation. For colony formation and differentiation assays, uncoated tissue culture plastic (BD, Falcon Primaria™, Allschwil, Switzerland) was used.

Colony formation and differentiation assays were performed in the medium according to the present invention. MSC were propagated in the medium according to the present invention or MSCM (DMEM/F12 supplemented with 15 mM HEPES, 10% fetal calf serum and 1× penicillin/streptomycin (Invitrogen/GIBCO, Basel, Switzerland)). For the colony formation and differentiation assays 1000 cells were seeded per well in 6 well plates in medium according to the present invention for 7 to 10 days prior to fixation. For differentiation of beta casein secreting alveolar cell lineage a method from (Dontu et al., 2003, Genes Dev 17, 1253-1270) was adapted. Briefly, PHMEC were subjected to colony formation assays in medium according to the present invention. After 10 days the formed colonies were overlaid with matrigel and incubated for 2 days before a differentiation medium (medium according to the present invention further comprising hydrocortisone 1 μg/ml, insulin 5 μg/ml and recombinant human prolactin 1 ug/ml (all from SIGMA)) was added to the culture. 50% of the differentiation medium was replaced with fresh medium every second day of culture. The cells were fixed after 7 days exposure to differentiation medium.

### Antibodies and Immuno-Fluorescence Immunohistochemistry.

Prior to immuno-fluorescent staining of cultured cells, samples were fixed with ice cold methanol/acetone (50/50 vol/vol) for 10 min at room temperature. The following primary antibodies were used: keratin 14 (RB-9020, 1:4000), keratin 18 (MS-142, 1:2000), keratin 19 (MS-198 1:1000) beta casein (MS-935 1:1000) (Thermo Scientific, Stehelin, Basel, Switzerland), keratin 8/18 (GP11, 1:500) (Fitzgerald, 01720 MA, USA). The cells were incubated at 4° C. over night with primary antibodies. Goat anti-mouse, goat anti-rabbit and goat anti-guinea pig secondary antibodies coupled to Alexa 488, 568 or 633 (Molecular Probes, Invitrogen 1:500) were used for detection. The cells were incubated with secondary antibodies for 30 min at room temperature. ProLong®Gold antifade (Molecular Probes, Invitrogen) was used for mounting.

### Mesenchymal Stem Cell Differentiation Assays

50000 first passage MSC from several donor samples were seeded in 6 well PRIMARA™ (BD, Falcon) dishes and cultured in MSCM over night. The medium was then exchanged with MSCM medium containing 5 μg/ml insulin, 1 μM dexamethasone, 0.5 μM isobutylmethylxanthine, 60 μM indomethacin and 10 nM 17-β-estradiol (SIGMA) for adipogenic differentiation or with 1 μM dexamethasone, 10 mM b-glycerophosphate, 100 μM ascorbic acid 2-phosphate and 10 nM 17-β-estradiol (SIGMA) for osteogenic differentiation. The cultures were maintained for 20 days and fixed with Formalin. Adipogenic differentiation was visualized by Oil red O 0.3% (SIGMA) in isopropanol 57% and H2O 40% to detect lipid. Alkaline phosphatase activity staining (SIGMAFAST™ BCIP®, SIGMA) was performed to assess osteogenic differentiation.

1. A composition comprising the components listed in Table I in an amount that will lead to the respective concentration specified in Table I for each of said component when said composition is diluted with water into 1× medium.

2. The composition of claim 1 wherein the components are in an amount that will lead to the respective concentration specified in Table II for each of said component when said composition is diluted with water into 1× medium.

3. The composition of claim 1 which is in a liquid form and is a chemically-defined medium suitable for culturing cells.

4. The composition of claim 3 wherein the cells are primary cells.

5. The composition of claim 3, wherein said composition is concentrated and wherein the concentration of all the components is increased by about the same factor.

6. The composition of claim 1 further comprising a solid support coated with a matrix.

7. The composition of claim 6 wherein said matrix comprises laminins and collagens.

8. The composition of claim 7 wherein said matrix comprises Matrigel® and collagen type I.

9. The composition of claim 6 wherein said support is coated with a composition comprising 10-20% of Matrigel® and about 10 g/ml of collagen type I.

10. The composition of claim 6 wherein the solid support is a mesh.

11. A method of culturing cells comprising the step of placing cells in contact with a composition according to claim 3.

12. A method of screening for agents influencing the differentiation of undifferentiated cells comprising the steps of contacting undifferentiated cells cultured according to claim 11 with an candidate agent; and comparing the differentiation state of the previously undifferentiated cells after said contact with said candidate agent with the differentiation state of undifferentiated cells cultured according to claim 11 which have not been contacted with said agent, wherein a difference in the differentiation state of the cells indicates that the agent is an agent influencing the differentiation of undifferentiated cells.

13. The method of claim 12 wherein the comparison is done by comparing the expression profile of the cell populations.

14. A kit comprising a composition according to claim 1, wherein some of the components are present in one of more containers.
